(19) United States
(12) Patent Application Publication
(10) Pub. No.: US 2026/0115424 A1
(43) Pub. Date: Apr. 30, 2026

(54) MEDICAL DEVICES, SYSTEMS, AND RELATED METHODS FOR TISSUE TREATMENT AND MANIPULATION

(71) Applicant: Boston Scientific Scimed, Inc., Maple Grove, MN (US)

(72) Inventors: Austin Grant JOHNSON, Worcester, MA (US); Chris SON, Worcester, MA (US)

(73) Assignee: Boston Scientific Scimed, Inc., Maple Grove, MN (US)

(21) Appl. No.: 19/366,041

(22) Filed: Oct. 22, 2025

Related U.S. Application Data
(60) Provisional application No. 63/711,446, filed on Oct. 24, 2024.

(51) Int. Cl. A61M 25/00; A61B 18/00; A61B 18/14; A61M 25/06

(52) U.S. Cl. CPC A61M 25/0074; A61M 25/0097; A61B 2018/00601; A61B 18/1492; A61M 2025/0681; A61M 2205/0266

(57) ABSTRACT

Medical devices, systems and related methods for tissue treatment and manipulation are described. An exemplary medical system includes device including a catheter, at least one arm, and one or more pull wires. The catheter having a central longitudinal axis and including a catheter lumen. The arm is positioned at a distal end of the catheter with a proximal end of arm pivotally connected to the catheter and a distal end of the arm forming a free end. The pull wire(s) extend to the distal end of the catheter. The arm includes a first configuration where the arm is generally parallel with the central longitudinal axis and a second configuration where the pull wire(s) urges the free end radially outward. The medical system further includes an accessory medical device including a shaft slidably received within the catheter lumen.

### CROSS-REFERENCE TO RELATED APPLICATION(S)

This application claims the benefit of priority to U.S. Provisional Application No. 63/711,446, filed on Oct. 24, 2024, which is incorporated by reference herein in its entirety.

### TECHNICAL FIELD

Various aspects of this disclosure generally relate to medical devices, systems, and related methods for tissue manipulation and treatment. In particular, aspects of this disclosure relate to medical devices for, for example, manipulating, resecting, grasping, and/or collecting tissue, such as, for example, electrosurgical knives.

### BACKGROUND

Medical devices, such as endoscopes or other suitable introduction devices, are employed for a variety of diagnostic and surgical procedures, such as endoscopy, laparoscopy, arthroscopy, gynoscopy, thoracoscopy, and cystoscopy, etc. Many of these procedures are carried out for purposes of tissue resection, which may include removal of tissue of an organ or a gland to treat tumors, infestations, and the like. In particular, such procedures may be carried out by inserting an insertion device into a patient's body through a surgical incision, or via a natural anatomical orifice (e.g., mouth, vagina, or rectum), and performing the procedure or operation.

Electrocautery knives, in particular, have been used in many medical procedures, including Endoscopic Mucosal Resection (EMR), Endoscopic Sub-mucosal Dissection (ESD), and Endoscopic Sub-mucosal Resection (ESR), polypectomy, mucosectomy, etc., for resecting tissue from a treatment site. While using electrocautery knives, or other instruments for resecting tissue, an operator may find it desirable to manipulate tissue at a treatment site to prevent damage to heathy tissues at or near the treatment site and to position unhealthy tissues at the treatment site such that they may be more easily resected with the electrocautery knife.

The medical devices, systems, and relative methods for tissue manipulation and treatment of the current disclosure may rectify some of the deficiencies described above or address other aspects of the art.

### SUMMARY

Each of the aspects disclosed herein may include one or more of the features described in connection with any of the other disclosed aspects.

The disclosure includes medical devices, systems, and related methods for tissue manipulation and treatment such as polyps and lesions within the gastrointestinal system. For example, the disclosure includes a medical system comprising a medical device and an accessory medical device. The medical device includes a catheter having a proximal end, a distal end, and a central longitudinal axis. The medical device further includes a catheter lumen extending from the proximal end to the distal end of the catheter. The medical device includes at least one arm at the distal end of the catheter. A proximal end of the at least one arm being pivotally connected to the distal end of the catheter and a distal end of the at least one arm forming a free end. The medical device further includes one or more pull wires extending from the proximal end of the catheter to the distal end of the catheter. The accessory medical device includes a shaft slidably received within the catheter lumen. The at least one arm includes a first configuration and a second configuration. In the first configuration, the at least one arm is generally parallel with the central longitudinal axis. In the second configuration, the one or more pull wire urges the free end of the at least one arm radially outward relative to the central longitudinal axis.

The medical device may include one or more of the following aspects. For example, the at least one arm may be a first arm and the medical system may further comprise a second arm at the distal end of the catheter. The distal end of the catheter may include a first slot and a second slot. The first slot and the second slot may each extend from the distal end of the catheter toward the proximal end of the catheter. Relative to the central longitudinal axis, the first slot may be positioned circumferentially opposite the second slot. Each of the first slot and the second slot may include a first channel and a second channel. The first channel may extend through a distal face of the catheter at the distal end. The second channel may extend through a side surface of the catheter. The first channel may be perpendicular to the central longitudinal axis and the second channel may be parallel to the central longitudinal axis. Further, the first channel may intersect with the second channel. The first slot may be configured to receive a first portion of the first arm and the second slot may be configured to receive a first portion of the second arm. A proximal portion of each slot may include a pin or pivot. The first portion of the first arm may be pivotally connected to the pin or pivot of the first slot. The first portion of the second arm may be pivotally connected to the pin or pivot of the second slot. The first arm and the second arm may each define a second portion. The second portion of the first arm may remain exterior to the first slot in the first configuration and the second configuration and the second portion of the second arm may remain exterior to the second slot in the first configuration and the second configuration. A lateral dimension of the respective second portions of the first arm and the second arm may be greater than a lateral dimension of the respective first portions of the first arm and the second arm. Pivoting of the first arm about the pin or pivot of the first slot may be independent from the pivoting of the second arm about the pin or pivot of the second slot. The distal end of the first arm may include a blunted end and a distal end of the second may include a blunted end. The first arm and the second arm may each include a shape-memory material. Remembered shapes of the first arm and the second arm may be straight.

In some examples, the medical device may further include a handle including a central lumen in communication with the catheter lumen. A distal end of the handle may include a knob, such as a Touhy-Borst knob. Fastening the knob may prevent axial movement of handle along a length of the shaft of the accessory medical device. Unfastening the knob may permit axial movement along the length of the shaft.

According to some aspects of this disclosure, each of the first arm and the second arm may include a tapered portion between the respective distal end and the respective proximal end. The one or more pull wires may include a first pull wire and a second pull wire. The first pull wire may be operably coupled to a first actuator of the medical device and the second pull wire may be operably coupled to a second actuator of the medical device. A distal end of the first pull wire may be fixed to the tapered portion of the first arm and a distal end of the second pull wire may be fixed to the tapered portion of the second arm.

This disclosure further includes a medical system comprising a medical device and an accessory medical device. The medical device includes a handle including an actuator, a catheter extending from a distal end of the handle, and an arm at a distal end of the catheter. The catheter includes a proximal end, a distal end, and a central longitudinal axis. The catheter further includes a catheter lumen extending from the proximal end to the distal end of the catheter. A first end of the arm is pivotally connected to the distal end of the catheter. The arm includes a first configuration where the arm is generally parallel to the central longitudinal axis and a second configuration where the arm is disposed at an angle relative to the central longitudinal axis. The accessory medical device includes a shaft including an electrode at a distal end. The shaft is slidably received within the catheter lumen and extendable relative to the catheter lumen such that the electrode protrudes distally beyond the distal end of the catheter lumen. Urging the actuator in a first direction transitions the arm toward the first configuration and urging the actuator in a second direction, opposite to the first direction, transitions the arm toward the second configuration.

According to some aspects of this disclosure, the medical system may further comprise a pull wire. The actuator may be operably connected to a proximal end of the pull wire. The distal end of the pull wire may be fixed to the arm. Longitudinal movement of the actuator may impart longitudinal movement upon the pull wire to urge the arm toward either the first configuration or the second configuration. The medical system may further comprise an insertion device. The insertion device may include a working channel. The catheter may be slidably received within the working channel. The catheter may include a sheath or coil extending from the proximal end of the catheter to the distal end of the catheter. The sheath or coil may permit rotation of the catheter within the working channel of the insertion device.

This disclosure includes a method of treating a treatment site. The method comprises: (i) delivering a medical device to the treatment site, (ii) extending and energizing an electrode to cut tissue at the treatment site, (iii) manipulating the tissue with an arm of the medical device, and (iv) further extending the electrode to cut tissue.

According to one or more aspects of this disclosure, the arm may be a first arm. The medical device may further include a second arm. The manipulating step may further include manipulating the tissue with the second arm.

### BRIEF DESCRIPTION OF THE FIGURES

The accompanying drawings, which are incorporated in and constitute a part of this specification, illustrate various aspects of this disclosure and together with the description, serve to explain the principles of the disclosure.

FIG. 1 depicts a side view of an exemplary medical system including an exemplary first medical device, an exemplary second medical device, and an exemplary third medical device with the second medical device extending through the third medical device and the third medical device extending through the first medical device, and FIG. 1 features a zoomed in, cutaway view of a distal end of the second medical device and the third medical device.

FIG. 2 depicts a perspective view of a proximal portion of the second medical system depicted in FIG. 1.

FIG. 3A depicts a perspective view of a distal portion of the medical system depicted in FIG. 1.

FIG. 3B depicts a cross-sectional view of a distal portion of the medical system depicted in FIG. 1.

FIG. 4A depicts a side view of a distal portion of another exemplary medical system, including distal portions of the first medical device and second medical device depicted in FIG. 1 and a distal portion of another exemplary third medical device extending through the first medical device and being in a first configuration.

FIG. 4B depicts a side view of the distal portion of the medical system of FIG. 4A and illustrates the third medical device extending through a lumen of the first medical device and being in a second configuration.

FIG. 5 depicts a side view of a proximal portion of an exemplary medical device of an exemplary medical system.

FIG. 6 depicts an exemplary flow chart for a method of treating a treatment site.

### DETAILED DESCRIPTION OF THE FIGURES

Particular aspects of the disclosure are described in greater detail below. The terms and definitions provided herein control, if in conflict with terms and/or definitions incorporated by reference.

The terms “proximal” and “distal” are used herein to refer to the relative positions of the components of exemplary medical devices. As used herein, “proximal” refers to a position relatively closer to the exterior of the body or closer to an operator using the medical device. In contrast, “distal” refers to a position relatively further away from the operator using the medical device, or closer to the interior of the body.

As used herein, the terms “comprises,” “comprising,” “including,” “includes,” “having,” “has,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.”

Further, relative terms such as, for example, “about,” “substantially,” “approximately,” etc., are used to indicate a possible variation of ±10% in a stated numeric value or range. As used herein, the phrase “based on” is understood to be equivalent to the phrase “based at least on,” unless indicated otherwise. The term “or” is used disjunctively, such that “at least one of A or B” includes, (A), (B), (A and A), (A and B), (B and B), etc. It will also be understood that, although the terms first, second, third, etc. are, in some instances, used herein to describe various elements, these elements should not be limited by these terms. These terms are only used to distinguish one element from another. For example, a first arm could be termed a second arm, and, similarly, a second arm could be termed a first arm, without departing from the scope of the various described embodiments. The first arm and the second arm are both arms, but they are not the same arm.

Although scopes and endoscopes are referenced herein for illustration purposes, it will be appreciated that the disclosure encompasses any suitable medical device configured to allow an operator to access and view internal body anatomy of a subject and/or to deliver medical instruments, such as, for example, biopsy forceps, graspers, baskets, snares, probes, scissors, capture/retrieval devices, lasers, and other tools, into the subject's body. The medical devices herein may be inserted into a variety of body lumens and/or cavities, such as, for example, the urinary tract or gastrointestinal tract. It will be appreciated that, unless otherwise specified, bronchoscopes, duodenoscopes, endoscopes, gastroscopes, endoscopic ultrasonography (“EUS”) scopes, colonoscopes, ureteroscopes, bronchoscopes, laparoscopes, cystoscopes, aspiration scopes, sheaths, catheters, or any other suitable delivery device may be used in connection with the features described herein.

This disclosure is drawn to medical devices, systems and related methods for tissue treatment and manipulation. An exemplary medical system may include a first medical device, such as an insertion device, a second medical device such as an electrocautery knife assembly, and a third medical device. The third medical device may include a catheter having bendable, rotatable, pivotable, and/or movable arms at a distal end for manipulating tissue. The catheter of the third medical device may be extended through a working channel of the insertion device and the shaft and electrode may be extended through the catheter. An operator may manipulate tissue at a treatment site with the arms of the catheter to appropriately position the tissue for resection with the electrode.

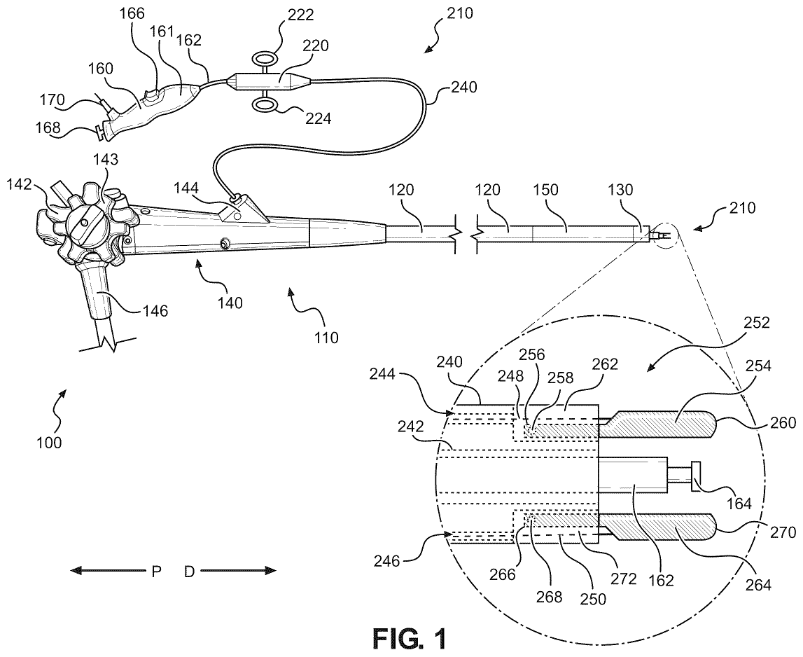

FIG. 1 depicts various aspects of an exemplary medical system 100, including an insertion device, herein a first medical device 110, an electrocautery knife assembly, herein a second medical device 160 (also referred to herein as an accessory medical device), and a third medical device 210. First medical device 110 includes handle 140 having a distal end and a shaft 120 extending distally from the distal end of handle 140. Shaft 120 includes a working channel 122. Second medical device 160 includes a handle 161 and a shaft 162 extending distally from a distal end of handle 161 to a distal end of shaft 162. The distal end of shaft 162 may include an electrode 164 that is extendable distally and retractable proximally relative to the distal end of shaft 162. Third medical device 210 may include a handle 220 and a catheter 240 extending from a distal end of handle 220. Catheter 240 may include one or more arms, for example, first arm 254 and second arm 264, at a distal end 252 of catheter 240 for manipulating, grasping, or cutting a tissue (e.g., a lesion) of a treatment site (e.g., the lesion and tissue surrounding the lesion).

As shown in FIG. 1, medical system 100 may be arranged such that shaft 162 of second medical device 160 is extended through handle 220 and catheter 240 of third medical device 210 to distal end 252 of catheter 240. Further, portions of second medical device 160 and third medical device 210 may be extended through first medical device 110. For example, catheter 240 and shaft 162 (e.g., including electrode 164) may be extended through a port (e.g., port 144) in communication with working channels 122 to a distal end 130 of shaft 120. After navigating to the treatment site, the operator may extend catheter 240 and/or electrode 164 beyond distal end 130 of shaft 120, such that arms 254, 264 are distal to distal end 130 and capable of pivoting or bending to manipulate (e.g., move, urge, grip, cut, tear, separate, pinch, etc.) tissue at the treatment site. Further, the operator may extend electrode 164 relative to catheter lumen 242 such that electrode 164 protrudes beyond distal end 252 of catheter 240 to deliver electrical energy to tissue of the treatment site.

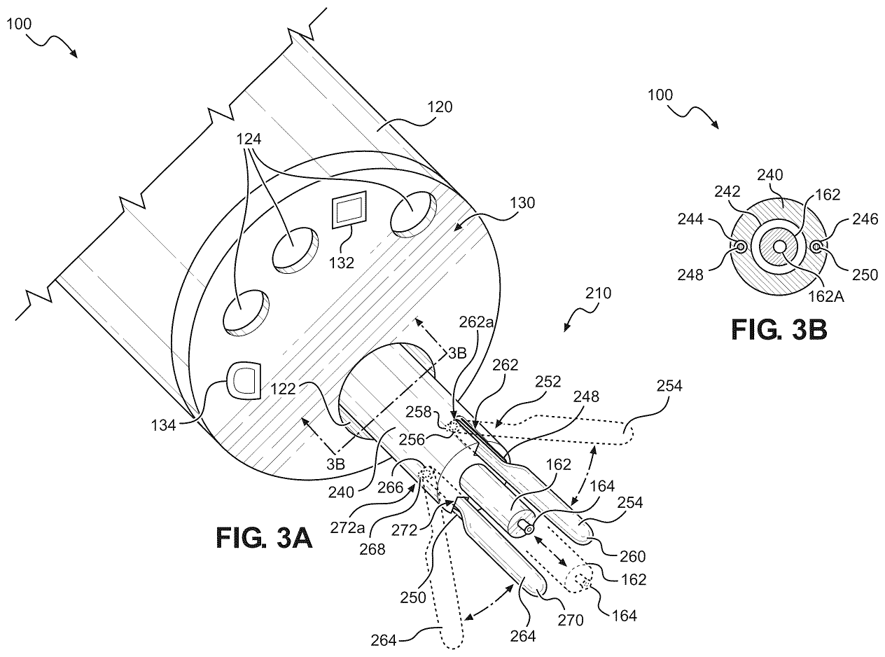

First medical device 110 is depicted in FIG. 1. First medical device 110 may be an endoscope, however this is merely exemplary and first medical device 110 may be any suitable delivery/insertion device. Medical device 110 includes shaft 120 having working channel 122. Shaft 120 may be generally tubular in shape and may include one or more channels extending from a proximal end of shaft 120 to distal openings (FIG. 3A) of the respective channels at distal end 130 of shaft 120. Working channel 122 may be configured to receive catheter 240 extended therethrough. Catheter 240 may include a sheath or coil extending from the proximal end of catheter 240 to the distal end of catheter 240. The sheath or coil may allow or permit rotation of catheter 240 along a central longitudinal axis of catheter 240 within working channel 122. Working channel 122 may include an opening at distal end 130 of shaft 120. As shown in FIG. 3A, shaft 120 may further include one or more channels 124, each of which may be configured to deliver fluid or provide negative pressure at openings of channels 124 at distal end 130. Handle 140 includes a port 144 in fluid communication with working channel 122. According to some aspects of this disclosure, handle may include additional ports, each in fluid communication with at least one of one or more channels 124 and may be connected to a vacuum source or fluid source (not shown).

Shaft 120 may be a flexible shaft (e.g., a catheter) having an articulation section 150 near distal end 130. One or more actuators, such as first actuator 142 and second actuator 143, may control articulation of articulation section 150. For example, the actuators 142, 143 may control movement of articulation section 150 in multiple directions, for example movement along different planes.

An umbilicus 146 of first medical device 110 may extend from handle 140 and may include or carry wires, cables, and/or conduits configured to provide, for example, power, signals, or fluids to and/or from handle 140. For example, umbilicus 146 may connect handle 140 to one or more user interfaces, monitors, controls units, displays, etc. Distal end 130 (e.g., a distal face of distal end 130) of shaft 120 may further include one or more illuminations devices or light 132 (e.g., LEDs, optical fibers (e.g., plastic optical fibers (POFs)), or other suitable light sources), and one or more imagers or camera 134. Umbilicus 146 may be connected to light 132 and/or cameras 134. Camera 134 may be configured to take video and/or still images, and may provide a signal to a monitor or display (e.g., connected via umbilicus 146).

Second medical device 160 includes handle 161 and shaft 162 extending from the distal end of handle 161. Shaft 162 may include a channel or lumen extending from a proximal end of shaft 162 to the distal end of shaft 162. Electrode 164 may be moveably or slidably received within the lumen of shaft 162. For example, electrode 164 may be axially movable relative to shaft 162 (e.g., longitudinally extendable and/or retractable longitudinally relative to the distal end of shaft 162). Electrode 164 may be energized to puncture, perforate, cut, resect, cauterize, remove, or otherwise treat tissue or other portions of a treatment site. According to some embodiments, electrode 164 may be fixed to the distal end of shaft 162. In some aspects, although not shown, electrode 164 may include an electrode lumen or a central lumen that terminates in an electrode outlet or electrode opening to inject or otherwise deliver fluid to the treatment site, for example, into or between layers of tissue. In other aspects, however, electrode 164 may not include an electrode lumen, and thus may not provide for injection or other delivery of fluid.

Handle 161 of second medical device 160 may include one or more actuations mechanisms such as a button, actuator, or trigger for example, movable relative to an exterior surface of handle 161. One or more actuation mechanisms may include a trigger 166. Trigger 166 may be movable, for example, slidable (e.g., longitudinally movable), actuatable, or depressible, to control a position (e.g., extension and/or retraction) and/or energization of electrode 164. For example, sliding, actuating, or depressing trigger 166 may extend electrode 164 from the distal end of shaft 162. According to some aspects where electrode 164 is fixed to the distal end of shaft 162, actuating trigger 166 may extend shaft 162 and electrode 164 distally beyond distal end 252 of catheter 240. Alternatively or additionally, in some aspects, trigger 166 (or another, different actuator) may control the energization of electrode 164. According to some aspects of this disclosure, the operator may extend and/or energize electrode 164 via trigger 166, one or more other actuators of handle 161, and/or one or more other actuators of handle 220.

Handle 161 also includes one or more connections and/or ports, for example, extending from or otherwise coupled to the exterior of handle 161. For example, handle 161 may include one or more of an electrode fluid port 168 and/or an electrical connection 170. Electrode fluid port 168 may be fluidly connected to electrode 164, for example, via one or more lumens in handle 161 and shaft 162. The one or more lumens in handle 161 and shaft 162 may include a fluid lumen 162A (FIG. 3B) extending through handle 161 and shaft 162 to electrode 164. Fluid lumen 162A may further extend through electrode 164 or be fluidly connected to a lumen of electrode 162. Electrode fluid port 168 may receive fluid (e.g., water, Orise™ gel, saline, etc.) to be delivered via a lumen (such as fluid lumen 162A) in electrode 164. Electrical connection 170 may be electrically connected to electrode 164, for example, via one or more conductive elements (e.g., wires, cables, filaments, rods, etc.) in handle 161 and shaft 162. Moreover, electrical connection 170 may receive energy (e.g., cautery energy) from an energy source to be delivered to electrode 164.

With reference to FIGS. 1-3, third medical device 210 may include handle 220 and catheter 240 extending from the distal end of handle 220. Catheter 240 may include first arm 254 and second arm 264 at distal end 252. First and second arms 254, 264 each include a first configuration, a second configuration, and intermediate/transitory configurations between the first configuration and the second configuration. Arms 254, 264 may be transitioned between the first configuration and the second configuration during an operation to manipulate tissue at the treatment site. The various configurations of arms 254, 264 are discussed in detail in proceeding sections of this disclosure.

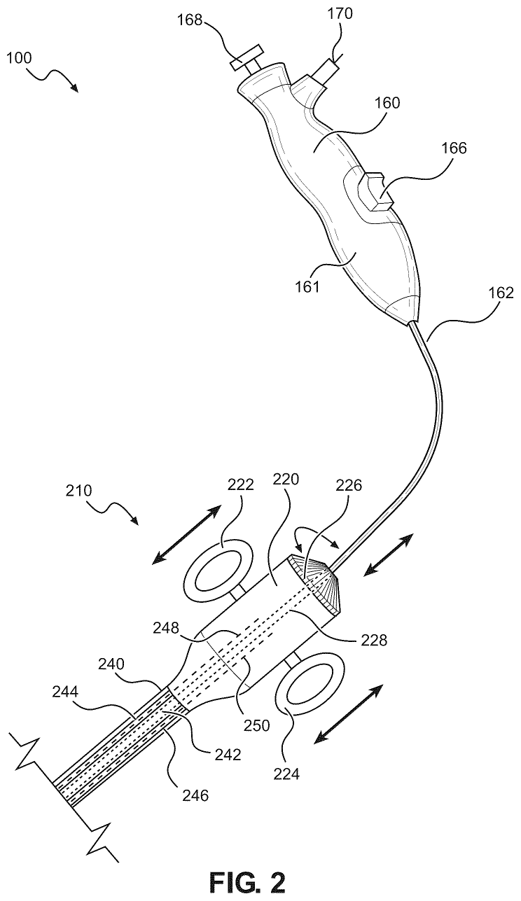

As shown in FIG. 2, handle 220 may include one or more actuators, for example, first actuator 222 and second actuator 224. Actuators 222, 224 may each include a lever, a slider, a knob, and other known actuators. For example, as shown in FIG. 2, actuators 222, 224 may each include a slider moveable relative to handle 220 in a proximal direction and a distal direction. For example, actuators 222, 224 may be longitudinal movable relative to the central longitudinal axis of catheter 240. Actuators 222, 224 may each include a ring at an end configured to receive a digit of an operator. Actuators 222, 224 each may be configured to manipulate, pivot, bend, move, or rotate one of first arm 254 and second arm 264, respectively, via one or more pull wires extending from handle 220 to distal end 252. For example, first actuator 222 may be operably connected to a proximal end of a first pull wire 248, and a distal end of the first pull wire 248 may be connected or fixed to first arm 254. Additionally, second actuator 224 may be operably connected to a proximal end of a second pull wire 250, and a distal end of second pull wire 250 may be connected or fixed to second arm 264. Each of first actuator 222 and second actuator 224 may be configured to transition one or more of arms 254, 264 between the first configuration and the second configuration. For example, longitudinal movement of actuators 222, 224 may impart longitudinal movement upon respective pull wires 248, 250 to urge respective arms 254, 264 toward either the first configuration or the second configuration as will be discussed below.

Still referring to FIG. 2, handle 220 may include a central lumen 228 configured to receive shaft 162 of second medical device 160 therethrough. Central lumen 228 may be in communication with a catheter lumen 242 (e.g., a central lumen) of catheter 240, such that shaft 162 may be extended through central lumens 228, 242 of handle 220 and catheter 240, respectively. A proximal end of handle 220 may include a knob 226 configured to fasten, secure, or otherwise couple handle 220 to shaft 162. For example, knob 226 may be a Touhy-Borst knob. Knob 226 may include a central opening sized and shaped to receive shaft 162 therethrough. While shaft 162 is extended through the central opening of knob 226, knob 226 may be rotated in a first direction to fasten handle 220 to shaft 162, and knob 226 may be rotated in a second direction, opposite to the first direction, to unfasten handle 220 to shaft 162. While not fastened to shaft 162, knob 226 may permit relative movement of handle 220 and shaft 162, for example, axial or longitudinal movement of one of handle 220 or shaft 162 relative to the other of handle 220 or shaft 162. Contrastingly, when handle 220 is fastened to shaft 162 via knob 226, handle 220 and shaft 162 may be fixed relative to one another, for example, in a fixed axial or longitudinal position along a length of shaft 162. Shaft 162 and electrode 164 may be extended distally or retracted proximally relative to distal end 252 of catheter 240 via axial or longitudinal movement of one of handle 220 or shaft 162 relative to the other of handle 220 or shaft 162. For example, shaft 162 and electrode 164 may be extended distally relative to distal end 252 of catheter 240 by unfastening knob 226 and moving handle 220 axially or longitudinally in the proximal direction relative to shaft 162. It follows that shaft 162 and electrode 164 may be retracted proximally relative to distal end 252 of catheter 240 by moving handle 220 axially or longitudinally in the distal direction relative to shaft 162.

As depicted in FIGS. 1 and 3A, catheter 240 includes first arm 254 and second arm 264. For example, first arm 254 and second arm 264 may be disposed at distal end 252 of catheter 240. Arms 254, 264 may be formed from metal, metal alloys, plastics, thermoset plastics, rubber, polymers, ceramics, and combinations thereof.

Plastics used may be insulating plastics configured to not conduct electricity or otherwise inhibit the transfer of electricity between arms 254, 264 and electrode 164. For example, each of arms 254, 264 may be formed from a metal and may include a plastic coating over the surface of the metal. Arms 254, 264 may each include a proximal portion 256, 266, respectively, and a distal portion 260, 270, respectively. Distal portion 260, 270 may include a distal end forming respective free ends. Further, the distal end of distal portions 260, 270 may each include one or more of a blunted end (as shown in FIG. 3A), a hooked end, a pronged end, and/or a pointed end. For example, distal portion 260 of first arm 254 may include a blunted end, and distal portion 270 of second arm 264 may include a hooked end. According to some aspects, arms 254, 264 and/or distal portions 260, 270 may each include a plurality of teeth or protrusions, for example, to help grip tissue.

A lateral dimension (e.g., a diameter or width) of distal portions 260, 270 may be greater than a lateral dimension of proximal portions 256, 266. In other words, a diameter of distal portions 260, 270 may be greater than a diameter of proximal portions 256, 266. Each of arms 254, 264 may include a tapered portion between respective proximal portions 256, 266 and respective distal portion 260, 270. The tapered portion may increase in diameter in the proximal to distal direction.

Each of proximal portions 256, 266 may each include a pin or pivot point 258, 268, respectively. For example, a proximal end of proximal portion 256 may include pin 258, and a proximal end of proximal portion 266 may include pin 268. As will be discussed in detail below, arms 254, 264 may be configured to pivot or rotate about pins 258, 268 (e.g., a longitudinal axis of pins 258, 268).

Referring to FIG. 1, catheter 240 may include one or more slots at distal end 252, each for receiving a portion of one of arms 254, 264 therein. For example, catheter 240 may include a first slot 262 for receiving at least a portion of proximal portion 256 of first arm 254, and catheter 240 may include a second slot 272 for receiving at least a portion of proximal portion 266 of second arm 264. In some embodiments including more than two arms, catheter 240 may include a corresponding number of slots. Slots 262, 272 may be positioned circumferentially opposite one another about distal end 252 of catheter 240. Each of slots 262, 272 may extend proximally from a first channel (or opening) defined through a distal face of catheter 240 at distal end 252. Further, each of slots 262, 272 may extend radially inward relative to the central longitudinal axis of catheter 240 from a second channel defined through a side surface or curved surface of catheter 240. The channels of slots 262, 272 defined through the distal face of catheter 240 may be generally perpendicular to the central longitudinal axis, and the channel defined through the side surface or curved surface of catheter 240 may be generally parallel with the central longitudinal axis. The first and second channels of each of respective slots 262, 272 may intersect at a perimeter or edge of the distal face of catheter 240.

Each of first slot 262 and second slot 272 may include an opening sized and shaped to receive the pin of one of respective arms 254, 264. For example, first slot 262 may include a first opening 262a, configured to receive first pin 258, at and/or near a proximal end of first slot 262 and second slot 272 may include a second opening 272a, configured to receive a second pin 268, at and/or near a proximal end of second slot 272. Pins 258, 268 may be configured to rotate about the longitudinal axis of respective pins 258, 268 within the first and second opening 262a, 272a of slots 262, 272. In some embodiments, instead of arms 254, 264 including respective pins 258, 268, slots 262, 272 may include one of pins 258, 268 and each of arms 254, 264 may include a corresponding opening configured to receive one of pins 258, 268.

Referring to FIGS. 1, 3A, and 3B, catheter 240 may include one or more lumens extending from a proximal end of catheter 240 to distal end 252. FIG. 3B depicts a cross section of catheter 240, and illustrates the one or more lumens of catheter 240. The one or more lumens of catheter 240 may include catheter lumen 242, a first pull wire lumen 244, and a second pull wire lumen 246. Catheter lumen 242 may be sized and shaped to receive shaft 162 and electrode 164 therethrough. Shaft 162 and electrode 164 may be movable or slidable relative to catheter lumen 242, such that the operator may extend shaft 162 and electrode 164 to distal end 252 via catheter lumen 242. Each of slots 262, 272 may be in communication with at least one of pull wire lumens 244, 246. For example, a distal opening of first pull wire lumen 244 may be defined through the proximal end (e.g., a distally-facing surface) of first slot 262 and in communication with first slot 262 and a distal opening of second pull wire lumen 246 may be defined through the proximal end of second slot 272.

First and second pull wire lumens 244, 246 may each be sized and shaped to receive a pull wire (e.g., or push wire) extending from handle 220 to distal end 252. For example, first pull wire lumen 244 may include first pull wire 248, and second pull wire lumen 246 may include second pull wire 250. Each of the first pull wire 248 and second pull wire 250 have a proximal end and a distal end. The proximal ends of first and second pull wires 248, 250 may be operably connected to actuators 222, 224, respectively, such that moving or urging actuators 222, 224 in a distal direction may slide or move pull wires 248, 250 in the distal direction (e.g., relative to catheter 240). Conversely, moving or urging actuators 222, 224 in a proximal direction may slide or move pull wires 248, 250 in the proximal direction. It should be understood that actuators 222, 224 may be configured to be moveable independent of each other and/or may be configured to be moveable in tandem, for example, based on one or more releasable connections (e.g., on or within handle 220).

As shown in at least FIG. 1, the distal end of first pull wire 248 may be operably connected first arm 254, and the distal end of second pull wire 250 may be operably connected to second arm 264. For example, first pull wire 248 and second pull wire 250 may be connected to the tapered portions of respective arms 254, 264. First pull wire 248 and second pull wire 250 may be connected to respective arms 254, 264 via welding, crimping, press fitting, an adhesive, etc. In some exemplary embodiments, each of arms 254, 264 may include a notch or groove configured to receive a portion of respective pull wires 248, 250 and couple the respective pull wire 248, 250 to arms 254, 264. According to some aspects of this disclosure, instead of first pull wire 248 and second pull wire 250 being connected to respective tapered portions of arms 254, 264, first and second pull wires 248, 250 may be connected to respective proximal portions 256, 266 or respective distal portions 260, 270 of arms 254, 264.

FIG. 3A illustrates exemplary configurations of arms 254, 264. For example, arms 254, 264 may be in the first configuration as shown in solid-line or in a second or transitory configuration as shown in dashed-line. The operator may independently actuate arms 254, 264 as necessary to manipulate the tissue such that the entire tissue or a portion of the tissue may be resected (e.g., via electrode 164). For example, moving pull wires 248, 250 via respective actuators 222, 224 relative to catheter 240 may transition first and second arms 254, 264 between the first configuration and the second configuration, as well as intermediate/transitory configurations between the first and second configurations. In the first configuration of each of arms 254, 264, a longitudinal axis of arms 254, 264 may be generally parallel with the central longitudinal axis of catheter 240 and/or distal ends of arms 254, 264 may be pointed generally toward the treatment site or otherwise in the same general direction as electrode 164. Contrastingly, in the second configuration of each of arms 254, 264, the longitudinal axis of arms 254, 264 may be generally angled away from the central longitudinal axis of catheter 240 and/or extend radially out or away from catheter 240 relative to the central longitudinal axis. According to some aspect of this disclosure, in the second configuration of each of arms 254, 264, may be generally perpendicular to the central longitudinal axis of catheter 240. In intermediate configurations between the first configuration and the second configuration, arms 254, 256 may be disposed at an angle, such as about zero degrees to about one hundred thirty-five degrees, relative to the central longitudinal axis of catheter 240. For example, when each of arms 254, 256 is in an intermediate configuration, first arm 254 may be disposed at a forty-five degree angle and second arm 256 may be disposed at a sixty degree angle. The respective distal portions 260, 270 of arms 254 may remain exterior to respective slots 262, 272 in the first configuration, the second configuration, and intermediate configurations.

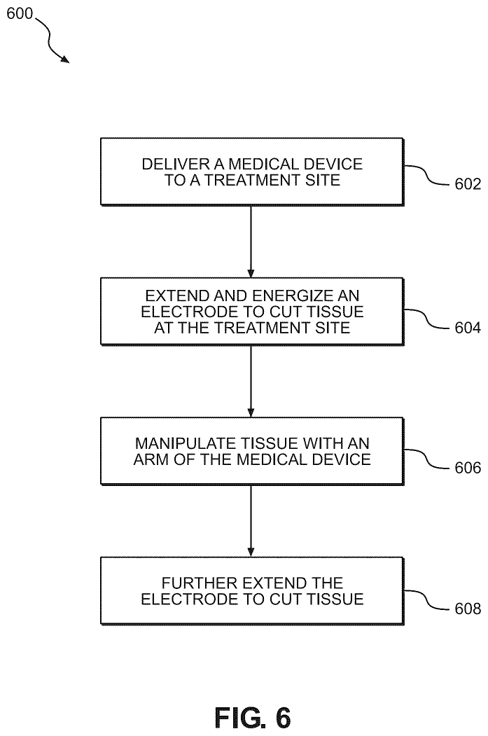

As shown in FIG. 6, this disclosure includes a method of treating a treatment site, a method 600. For example, medical system 100, as well as the other medical systems and devices disclosed in this disclosure, may be used with method 600 in an exemplary medical procedure (e.g., endoscopic submucosal dissection) at a treatment site within a body lumen or orifice of a subject. Method 600 may include initial steps pertaining to assembly of the medical system. For example, method 600 may include an initial step of inserting catheter 240 into port 144 and extending catheter 240 through working channel 122 to distal end 130 of shaft 120. Further, method 600 may include an initial step of inserting shaft 162 of second device 160 through handle 220 and catheter lumen 242 of catheter 240, and extending shaft 162 through catheter lumen 242 until electrode 164 is at or proximate distal end 252.

Method 600 may include an initial step of navigate distal end 130 of shaft 120 through a body lumen so that distal end 130 is proximate the treatment site. The operator may deflect articulation section 150 of shaft 120 by actuating an actuator (e.g., actuators 142, 143) of medical device 110.

Method 600 may include a step 602 of delivering a medical device to a treatment site. For example, after navigating distal end 130 of shaft 120 to the treatment site, the operator may extend (e.g., deliver) catheter 240 of third medical device 210 distally relative to working channel 122, such that distal end 252 of catheter 240 is distal to distal end 130 of shaft 120 and at least one of arms 254, 264 is capable of manipulating the tissue at the treatment site. Shaft 162 and electrode 164 of second medical device 160 may be disposed within catheter lumen 242 and between arms 254, 264. For example, shaft 162 and electrode 164 may extended through catheter lumen 242 to distal end 252 of catheter 240 and/or distally beyond distal end 252 such that electrode 164 protrudes beyond the distal face of catheter 240.

Method 600 may further include a step 604 of extending and energizing an electrode to cut tissue at the treatment site. For example, the operator may actuate trigger 166 to extend electrode 164 distally relative to the distal end of shaft 162 and/or distal end 252 of catheter 240. Electrode 164 may cut through tissue at the treatment site upon contacting said tissue. Step 604 may further include a step of retracting and de-energizing electrode 164 after making an initial cut through the tissue. For example, the operator may extend and/or energize electrode 164 via actuating trigger 166, one or more other actuators of handle 161, and/or one or more other actuators of handle 220 and may retract and/or de-energize electrode 164 by deactuating trigger 166, one or more other actuators of handle 161, and/or one or more other actuators of handle 220. In an exemplary embodiment, electrode 164 may be distally extended beyond respective distal ends of distal portions 260, 270 of arms 254, 264.

In addition to, or instead of, extending and retracting electrode 164 via trigger 166, the operator may extend and retract one or more of electrode 164 and distal end 252 of catheter 240 (e.g., arms 254, 264) via movement of one or more of handle 161 and handle 220. For example, the operator may distally extend electrode 164 relative to catheter 240 (e.g., distal end 252) by unfastening knob 226 and moving handle 161 distally to urge shaft 162 in the distal direction. Further, while knob 226 is unfastened, handle 161 may be moved in the proximal direction to urge shaft 162 proximally and retract electrode 164 relative to catheter 240. In some instances, it may be desirable to distally extend electrode 164 and arms 254, 264 relative to working channel 122 (e.g., distal end 130) so that electrode 164 or arms 254, 264 may engage with more tissue or different portions of tissue at the treatment site without requiring movement of shaft 120 relative to the treatment site. For example, shaft 162 and catheter 240 may be urged in the distal direction relative to working channel 122 to distally extend electrode 164 and arms 254, 264 by moving one or more of handle 161 and handle 220 in the distal direction while knob 226 is fastened or by moving both of handle 161 and handle 220 in the distal direction while knob 226 is fastened or unfastened. Conversely, electrode 164 and arms 254, 264 may be proximally retracted relative to working channel 122 by moving one or more of handle 161 and handle 220 in the proximal direction while knob 226 is fastened or by moving both of handle 161 and handle 220 in the proximal direction while knob 226 is fastened or unfastened.

Method 600 may include a step 606 of manipulating tissue with an arm of the medical device. For example, the operator may actuate actuators 222, 224 to rotate or pivot respective arms 254, 264 about respective pins 258, 268 (e.g., transitioning arms 254, 264 between the first and second configurations), such that distal ends of arms 254, 264 manipulate the tissue. The operator, using arms 254, 264, may manipulate the tissue to be an appropriate position for additional cutting via electrode 164. For example, actuating one or more of arms 254, 264 may urge a portion of the tissue away from the treatment site so that the operator may more easily cut away the tissue or otherwise treat other portions of the treatment site. In another example, actuating one or more of arms 254, 264 may mechanically separate, tear, or cut tissue at the treatment site, so that the operator cut, manipulate, or otherwise treat tissue with one or more of arms 254, 264 or electrode 164.

Method 600 may further include a step 608 of further extending the electrode to cut tissue. For example, after manipulating the tissue via arms 254, 264, the operator may further distally extend and energize electrode 164 via trigger 166, one or more other actuators of handle 161, and/or one or more other actuators of handle 220 to cut additional portions of the tissue at the treatment site. For example, while knob 226 is unfastened, the operator may urge shaft 162 distally relative to catheter 240 by moving handle 161 in the distal direction to further extend electrode 164, or may urge shaft 162 and catheter 240 distally relative to working channel 122 by moving both of handle 161 and handle 220 in the distal direction to further extend electrode 164 and arms 254, 264. While knob 226 is fastened, the operator may distally move one or more of handle 161 and handle 220 to further distally extend electrode 164 and arms 254, 264 relative working channel 122. When electrode 164 and/or arms 254, 264 is no longer needed, the operator may proximally retract electrode 164 and/or arms 254, 264 as discussed above. Method 600 may be repeated as necessary. For example, the operator may repeat the above-mentioned steps as needed to remove additional tissues from the treatment site or additional portions of the tissue from the treatment site.

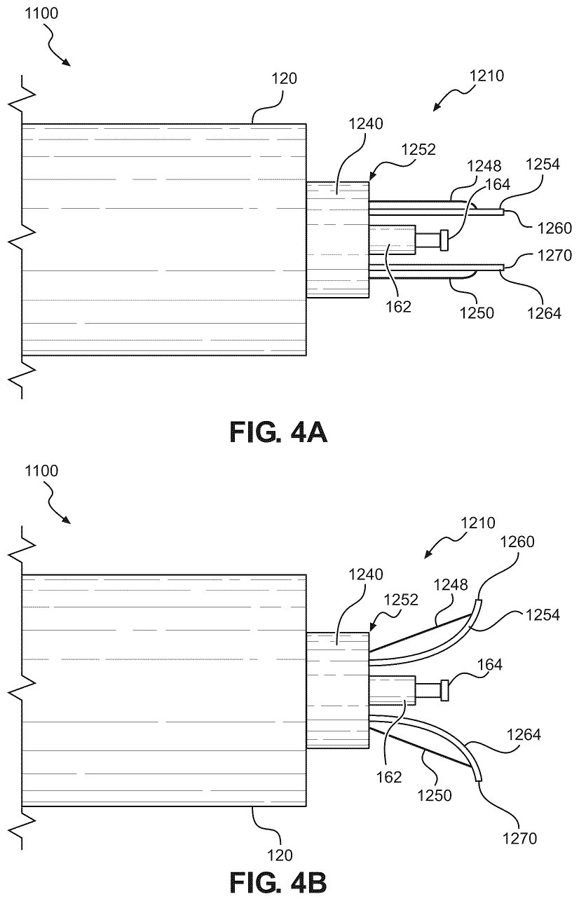

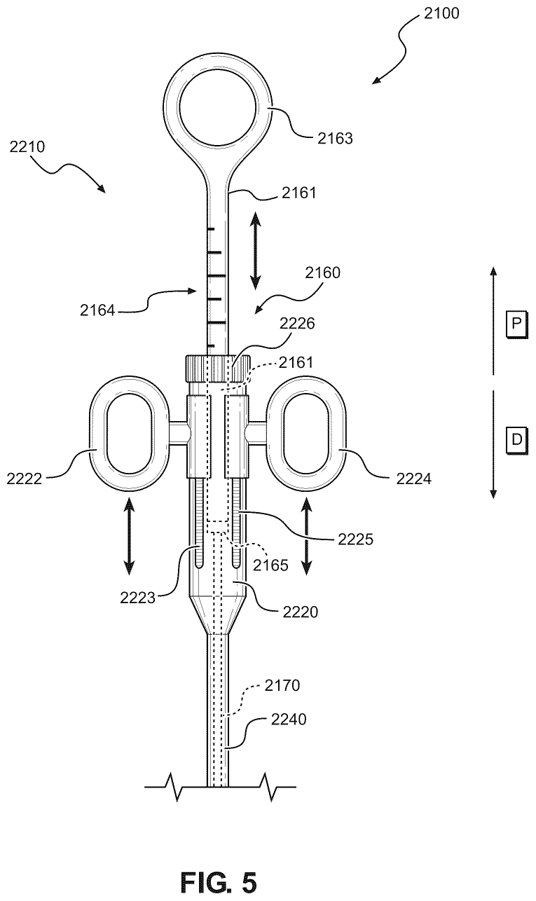

FIGS. 4A, 4B, and 5 illustrate additional medical devices according to some aspects of this disclosure. For example, FIGS. 4A-4B illustrate an alternative embodiment of third medical device 210, a third medical device 1210, and FIG. 5 illustrates alternative embodiment including features of second medical device 160 and third medical device 210, a medical device 2210. Third medical device 1210 or medical device 2210 may be included in medical system 100 and may be used to perform one or more aspects of method 600.

FIGS. 4A-4B depict an alternative medical system to medical system 100, an exemplary medical system 1100. Medical system 1100 may include any and all of the features of medical system 100 unless otherwise specified. For example, medical system 1100 may include first medical device 110 and second medical device 160 as described above. Medical system 1100 may include third medical device 1210. Third medical device 1210 may be substantially similar to or identical to medical device 210 except the differences here described. Third medical device 1210 may include a handle (not shown) having one or more actuators and a catheter 1240 extending distally from the handle.

FIGS. 4A-4B show third medical device 1210 and shaft 120 of first medical device 110. In particular, FIGS. 4A-4B illustrate a distal end 1252 of catheter 1240 extending from distal end 130 of shaft 120 of first medical device 110. Third medical device 1210 may include a first arm 1254 and a second arm 1264. Arms 1254, 1264 may include any of the features of arms 254, 264, respectively. Arms 1254, 1264 may be formed from a flexible material and be configured to flex or deflect along a length of respective arms 1254, 1264. For example, arms 1254, 1264 may be formed from a shape-memory material such as Nitinol®, other nickel-titanium alloys, and other materials with shape-memory properties. Arms 1254, 1264 may include a remembered shape that is straight and/or linear. Instead of slots 262, 272, arms 1254, 1264 may extend distally from a distal face of catheter 1240. For example, the distal face of catheter 1240 may include one or more holes for receiving a proximal portion of one of arms 1254, 1264. Arms 1254, 1264 may be cantilevers with respect to the distal face of catheter 1240.

Third medical device 1210 may include one or more pull wires within one or more pull wire lumens extending from a proximal end of catheter 1240 to a distal end of catheter 1240. For example, catheter 1240 may include a first pull wire 1248, within a first pull wire lumen, operably connected to first arm 1254, and catheter 1240 may also include a second pull wire 1250, within a second pull wire lumen, operably connected to second arm 1264. Instead of distal openings of the first pull wire lumen and the second pull wire lumen being in communication with slots 262, 272, the distal openings of the first and second pull wire lumens may be defined through the distal face of catheter 1240. The first and second pull wire lumens may be positioned radially outward of arms 1254, 1264, relative to a central longitudinal axis of catheter 1240. Further, first pull wire 1248 may be connected to a portion of first arm 1254, and second pull wire 1250 may be connected to a portion of second arm 1264. For example, first and second pull wires 1248, 1250 may be connected to respective intermediate or distal portions 1260, 1270 of arms 1254, 1264 to facilitate deflections of arms 1254, 1264 when actuating pull wires 1248, 1250.

As shown in FIGS. 4A-4B, arms 1254, 1264, instead of pivoting about a pin within a slot, may be configured to bend or deflect in response to the operator extending distally and retracting proximally first and second pull wires 1248, 1250. Arms 1254, 1264 may each include a deflected configuration and an undeflected configuration. In the undeflected configuration, arms 1254, 1264 may be in their remembered shape and may be generally parallel with the central longitudinal axis of catheter 1240 or otherwise point in the same direction as electrode 164. In the deflected configuration, arms 1254, 1264 may include a radius of curvature and may be configured to deflect away from the central longitudinal axis. For example, the operator may independently actuate the one or more actuators (e.g., urging one or more actuators proximally) of the handle of third medical device 1210 to proximally retract pull wires 1248, 1250. Proximally retracting pull wires 1248, 1250 may cause arms 1254, 1264 to transition from the undeflected configurations to the deflected configurations, such that respective distal portions 1260, 1270 of arms 1254, 1264 bend away from the central longitudinal axis of catheter 1240. The operator may increase the radius of curvature of arms 1254, 1264 by further actuating the one or more actuators (e.g. further urging one or more actuators proximally). The operator may actuate the one or more actuators (e.g., urging one or more actuators distally) to distally extend pull wires 1248, 1250. Distally extending pull wires 1248, 1250 may cause arms 1254, 1264 to transition from the deflected configurations to the undeflected configurations, such that respective distal portions 1260, 1270 of arms 1254, 1264 are transition toward the central longitudinal axis of catheter 1240 until arms 1254, 1264 are generally parallel with the central longitudinal axis of catheter 1240. Alternatively, instead of actuating the one or more actuators to transition arms 1254, 1264 to transition from the deflected configurations to the undeflected configurations, the operator may release the one or more actuators so that arms 1248, 1250 may automatically transition to their remembered shapes due to the shape-memory properties of arms 1248, 1250.

According to some aspects of this disclosure, arms 1254, 1264 may further include an inwardly-deflected configuration. For example, from the undeflected or deflected configurations, the operator may actuate the one or more actuators (e.g., urging one or more actuators distally) to transition arms 1254, 1264 to the inwardly-deflected configuration such that first and second pull wires 1248, 1250 urge respective distal portions 1260, 1270 of arms 1254, 1264 to bend or otherwise move toward and be angled toward the central longitudinal axis of catheter 1240.

FIG. 5 depicts an alternative medical system to medical systems 100, 1100, an exemplary medical system 2100. Medical system 1200 may include any and all of the features of medical system 100 unless otherwise specified. Medical system 2100 may include a medical device 2210. Medical device 2210 may include any of the features of second medical device 160 and third medical devices 210, 1210. Medical device 2210 may include a handle 2220. Medical device 2210 may include a catheter 2240 extending distally from a distal end of handle 2220. Catheter 2240 may include any of the features of catheters 240, 1240. For example, a distal end of catheter 2240 may include any of the features of distal end 252 of catheter 240, such as slots 262, 272 and arms 254, 264. Alternatively, in replacement or in additional to the features of catheter 240, the distal end of catheter 2240 may include any of the features of distal end 1252 of catheter 1240 such as arms 1254, 1264.

Handle 2220 may include one or more actuators each operably coupled to one or more pull wires that are operably connected to one or more arms at the distal end of catheter 2240. For example, handle 2220 may include a first actuator 2222 and a second actuator 2224. Handle 2220 may include a first slot 2223 configured to receive a portion of first actuator 2222 extended therethrough, and handle 2220 may also include a second slot 2225 configured to receive a portion of second actuator 2224 extended therethrough. Slots 2223, 2225 may be defined through an exterior surface of handle 2220. First and second actuators 2222, 2224 may be slidable in the proximal and distal directions along respective slots 2223, 2225. For example, the one or more pull wires of handle 2220 may be distally extended by sliding one or more of actuators 2222, 2224 in the distal direction and may be proximally retracted by sliding one or more of actuators 2222, 2224 in the proximal direction.

Handle 2220 may include a central lumen in communication with a central lumen of catheter 2240. Further, medical device 2210 may include a shaft assembly 2160 including a proximal handle or rod 2161 and a distal shaft 2170. At least a portion of rod 2161 may extend through the central lumen of handle 2220. For example, rod 2161 may include a proximal portion positioned proximal to a proximal end of handle 2220 and a distal portion positioned within the central lumen of handle 2220. At least a portion of shaft 2170 may extend through the central lumen of handle 2220 and through the central lumen of catheter 2240. For example, shaft 2170 may include a proximal portion positioned within the central lumen of handle 2220 and a distal portion positioned at or near the distal end of catheter 2240. The distal end of shaft 2170 may include an electrode (e.g., electrode 164). As shown in FIG. 5, rod 2161 may include a ring 2163, for example, positioned at the proximal end of rod 2161. Ring 2163 may configured to receive a digit (e.g., a thumb) of the operator. The distal end of rod 2161 may be releasably connected or fixedly attached to the proximal end of shaft 2170. For example, the proximal end of shaft 2170 may include a connector 2165. The distal end of rod 2161 may be connected to shaft 2170 via connector 2165. According to some aspects, the distal end of rod 2161 may include a first connector component, and the proximal end of shaft 2170 may include a corresponding second connector component configured to be mated with the first connector component to releasably attach rod 2161 to shaft 2170.

When rod 2161 is connected to shaft 2170 via connector 2165, moving rod 2161 in a direction may urge shaft 2170 to slide within the catheter lumen of catheter 2240 in the same direction. For example, rod 2161 may be moved (e.g., pushed) in the distal direction to urge shaft 2170 to slide distally within the catheter lumen until electrode 164 is distal to the distal end of catheter 2240. Conversely, rod 2161 may be moved (e.g., pulled) in the proximal direction to urge shaft 2170 to slide proximally within the catheter lumen until electrode 164 is proximal to the distal end of catheter 2240. In other words, moving rod 2161 may axially extend or retract shaft 2170 within the central lumens of handle 2220 and catheter 2240.The operator may axially extend shaft 2170 relative to handle 2220 and catheter 2240, such that electrode 164 of shaft 2170 extends distally beyond the distal end of catheter 2240 to deliver treatment to the treatment site.

Handle 2220 may include a knob 2226 at a proximal end of handle 2220. Knob 2226 may be a Touhy-Borst knob. Knob 2226 may include an opening configured to receive a portion of shaft assembly 2160 (e.g., rod 2161 and/or shaft 2170) therethrough. Rotating knob 2226 in a first direction may fasten, secure, or otherwise couple handle 2220 to rod 2161, in order to fix an axial position of handle 2220 relative to shaft assembly 2160. Further, rotating knob 2226 in a second direction, opposite to the first direction, may unfasten, unsecure, or otherwise uncouple handle 2220 to rod 2161 such that shaft assembly 2160 may be slid or otherwise moved axially relative to handle 2220 in the proximal and distal directions. Although not shown, one or more portions of medical device 2210 may include one or more ports, for example, to deliver fluid longitudinally through the electrode. Additionally or alternatively, although not shown, one or more portions of medical device 2210 may include one or more energy hubs, for example, to receive energy to energize the electrode.

Still referring to FIG. 5, shaft assembly 2160 may include a plurality of indicia 2164. For example, plurality of indicia 2164 may be disposed along a length of rod 2161 between ring 2164 and the distal end of rod 2161 Plurality of indicia 2164 may be configured to measure, designate, specify, indicate, or otherwise correlate a position of rod 2161 relative to handle 2220 to a distance between the distal end of catheter 2240 and electrode 164. Plurality of indicia 2164 may include a common length or offset between adjacent indicia of plurality of indicia 2164 of about 0.1 mm to about 50 mm, for example the common length may be 5 mm. In other words, adjacent indicia of plurality of indicia 2164 may be separated by a common distance. While the common length or offset of 5 mm is described herein and depicted in FIG. 5, the disclosure is not so limited. Rather, the common length between adjacent indicia of plurality of indicia 2164 may be any appropriate length (e.g., 0.5 mm, 1 mm, 5 mm, 10 mm, 20 mm, 25 mm, or fractions therebetween, etc.) to facilitate measurement of the distance between electrode 164 and the distal end of catheter 2240. The operator may measure the distance between the distal end of catheter 2240 and electrode 164 by counting the number of indicia between a proximal end of plurality of indicia 2164 and the proximal end of knob 2226. For example, where the plurality of indicia 2164 includes five indicia each with a length of 1 mm, the operator may count the number of indicia proximal to the proximal end of knob 2226 to determine the distance between electrode 164 and knob 2226. If two indicia of the five indicia of plurality of indicia 2164 are proximal to proximal end of knob 2226, electrode 164 may be positioned approximately 3 mm distal to the distal end of catheter 2240. Moreover, the length of an indicia of plurality of indicia 2164 may be measured with any unit of length such as, but not limited to, inches (in), centimeters (cm), etc. Plurality of indicia 2164 may include one or more sets of indicia measured in one or more units. For example, plurality of indicia 2164 may include a first set of indicia measured in a first unit, such as centimeters, and a second set of indicia in a second unit, such as inches.

While principles of this disclosure are described herein with reference to illustrative examples for particular applications, it should be understood that the disclosure is not limited thereto. Those having ordinary skill in the art and access to the teachings provided herein will recognize additional modifications, applications, embodiments, and substitution of equivalents all fall within the scope of the features described herein. Accordingly, the claimed features are not to be considered as limited by the foregoing description.

What is claimed is:

1. A medical system comprising:
a medical device including:
a catheter having a proximal end, a distal end, and a central longitudinal axis, and further including a catheter lumen extending from the proximal end to the distal end of the catheter,
at least one arm at the distal end of the catheter, a proximal end of the at least one arm pivotally connected to the distal end of the catheter, and a distal end of the at least one arm forming a free end, and
one or more pull wires extending from the proximal end of the catheter to the distal end of the catheter; and
an accessory medical device including a shaft slidably received within the catheter lumen;
wherein the at least one arm includes a first configuration and a second configuration;
wherein, in the first configuration, the at least one arm is generally parallel with the central longitudinal axis;
wherein, in the second configuration, the one or more pull wires urges the free end of the at least one arm radially outward relative to the central longitudinal axis.

2. The medical system of claim 1, wherein the at least one arm is a first arm, and wherein the medical system further comprises: a second arm at the distal end of the catheter, wherein the distal end of the catheter includes a first slot and a second slot, wherein the first slot and the second slot each extend from the distal end of the catheter toward the proximal end of the catheter, wherein, relative to the central longitudinal axis, the first slot is positioned circumferentially opposite to the second slot.

3. The medical system of claim 2, wherein each slot of the first slot and the second slot includes a first channel and a second channel, wherein the first channel extends through a distal face of the catheter at the distal end, wherein the second channel extends through a side surface of the catheter, wherein the first channel is perpendicular to the central longitudinal axis and the second channel is parallel to the central longitudinal axis, and wherein the first channel intersects with the second channel.

4. The medical system of claim 2, wherein the first slot is configured to receive a first portion of the first arm, and wherein the second slot is configured to receive a first portion of the second arm.

5. The medical system of claim 4, wherein a proximal portion of each slot includes a pin or pivot, wherein the first portion of the first arm is pivotally connected to the pin or pivot of the first slot, and wherein the first portion of the second arm is pivotally connected to the pin or pivot of the second slot.

6. The medical system of claim 5, wherein the first arm and the second arm each define a second portion, wherein the second portion of the first arm remains exterior to the first slot in the first configuration and the second configuration, and wherein the second portion of the second arm remains exterior to the second slot in the first configuration and the second configuration.

7. The medical system of claim 5, wherein a lateral dimension of the respective second portions of the first arm and the second arm is greater than a lateral dimension of the respective first portions of the first arm and the second arm.

8. The medical system of claim 5, wherein pivoting of the first arm about the pin or pivot of the first slot is independent from pivoting of the second arm about the pin or pivot of the second slot.

9. The medical system of claim 2, wherein the medical device further comprises a handle, wherein the handle includes a central lumen in communication with the catheter lumen, wherein a distal end of the handle includes a knob, wherein fastening the knob prevents axial movement of handle along a length of the shaft of the accessory medical device, and wherein unfastening the knob permits axial movement along the length of the shaft.

10. The medical system of claim 9, wherein the knob is a Touhy-Borst knob.

11. The medical system of claim 2, wherein the distal end of the first arm includes a blunted end and a distal end of the second arm includes a blunted end.

12. The medical system of claim 2, wherein each of the first arm and the second arm include a tapered portion between the respective distal end and the respective proximal end.

13. The medical system of claim 12, wherein the one or more pull wires includes a first pull wire and a second pull wire, wherein the first pull wire is operably coupled to a first actuator of the medical device, and wherein the second pull wire is operably coupled to a second actuator of the medical device.

14. The medical system of claim 13, wherein a distal end of the first pull wire is fixed to the tapered portion of the first arm, and wherein a distal end of the second pull wire is fixed to the tapered portion of the second arm.

15. The medical system of claim 2, wherein the first arm and the second arm each include a shape-memory material, wherein remembered shapes of the first arm and the second arm are straight.

16. A medical system comprising:
a medical device including:
a handle including an actuator;
a catheter extending from a distal end of the handle, wherein catheter includes a proximal end, a distal end, and a central longitudinal axis, and wherein the catheter includes a catheter lumen extending from the proximal end to the distal end of the catheter;
an arm at the distal end of the catheter, wherein a first end of the arm is pivotally connected to the distal end of the catheter, wherein the arm includes a first configuration where the arm is generally parallel to the central longitudinal axis and a second configuration where the arm is disposed at an angle relative to the central longitudinal axis; and
an accessory medical device including a shaft, wherein a distal end of the shaft includes an electrode;
wherein the shaft is slidably received within the catheter lumen and extendable relative to the catheter lumen such that the electrode protrudes distally beyond the distal end of the catheter lumen;
wherein urging the actuator in a first direction transitions the arm toward the first configuration and urging the actuator in a second direction, opposite to the first direction, transitions the arm toward the second configuration.

17. The medical system of claim 16, further comprising: a pull wire, wherein the actuator is operably connected to a proximal end of the pull wire, wherein the distal end of the pull wire is fixed to the arm, and wherein longitudinal movement of the actuator imparts longitudinal movement upon the pull wire to urge the arm toward either the first configuration or the second configuration.

18. The medical system of claim 16, further comprising: an insertion device, wherein the insertion device includes a working channel, wherein the catheter is slidably received within the working channel, wherein the catheter includes a sheath or coil extending from the proximal end of the catheter to the distal end of the catheter, and wherein the sheath or coil permits rotation of the catheter within the working channel of the insertion device.

19. A method of treating a treatment site, comprising:
delivering a medical device to the treatment site;
extending and energizing an electrode to cut tissue at the treatment site;
manipulating the tissue with an arm of the medical device; and
further extending the electrode to cut tissue.

20. The method of claim 19, wherein the arm is a first arm, wherein the medical device further includes a second arm, wherein the manipulating step further includes manipulating the tissue with the second arm.
